(19) United States
(12) Patent Application Publication
(10) Pub. No.: US 2003/0118646 A1
(43) Pub. Date: Jun. 26, 2003

(54) (R, S'),(R, R')-amphetaminil, compositions and uses thereof

(76) Inventors: Seth Lederman, New York, NY (US); Steve Leventer, Langhorne, PA (US); Robert Kucharik JR., Glenmoore, PA (US)

Correspondence Address: KLAUBER & JACKSON, 411 HACKENSACK AVENUE, HACKENSACK, NJ, 07601

(21) Appl. No.: 09/992,235

(22) Filed: Nov. 6, 2001

Related U.S. Application Data
(60) Provisional application No. 60/297,386, filed on Jun. 11, 2001.

(51) Int. Cl. A61K 9/22; A61K 31/277

(52) U.S. Cl. 424/468; 514/523

(57) ABSTRACT

Pharmaceutical compositions comprising the active (R,R′),(R,S′) form of amphetaminil substantially free of (S,R′),(S,S′)-amphetaminil are described for the treatment of humans.

### CROSS-REFERENCE TO RELATED APPLICATION

Priority is claimed under 35 U.S.C. §119(e) to Provisional Application Serial No. 60/297,386, filed Jun. 11, 2001, which is incorporated herein by reference in its entirety.

### BACKGROUND OF THE INVENTION

Amphetaminil, or alpha-[(1-methyl-2-phenylethyl)amino]benzeneacetonitrile, also known under the trademarks AN1 and APONEURON, is a neuropharmacologically-active compound that has been marketed for the treatment of narcolepsy, for use as a tonic, and for hypotension in combination with other components. One study has indicated potential utility in attention-deficit hyperactivity disorder (ADHD).

The amphetaminil molecule possesses two dissymmetric centers resulting in four possible enantiomers: (R,R′)-amphetaminil; (S,S′)-amphetaminil; (R,S′)-amphetaminil; and (S, R′)-amphetaminil. The commercially-available form (AN1) is a racemate, having a ratio of the (R,R′),(S,S′) diastereomers to the (R,S′),(S,R′) diastereomers of about 4-5:1 (Salvesen et al., 1974, Arzneim-Forschung (Drug Research) 24:137-140). However, amphetaminil that is substantially enantiomerically pure at the first dissymetric center (pure R: (R,S′),(R,R′)-amphetaminil, also referred to as alpha-[(1R-Methyl-2-phenylethyl)amino]benzeneacetonitrile; pure S: (S,R′),(S,S′)-amphetaminil, also referred to as alpha-[(1S-Methyl-2-phenylethyl)amino]benzeneacetonitrile) has not been prepared or evaluated for pharmacological activity.

It is towards identifying pharmacological activity in optical isomers of amphetaminil and improved utility of such isomers that the present invention is directed.

The citation of any reference herein should not be construed as an admission that such reference is available as “Prior Art” to the instant application.

### SUMMARY OF THE INVENTION

In one broad aspect, the invention is directed to a pharmaceutical composition containing at least an effective amount of (R,R′),(R,S′)-amphetaminil sulfate or another pharmaceutically-acceptable salt thereof, substantially free of (S,R′),(S,S′)-amphetaminil, and at least one pharmaceutically-acceptable carrier, diluent, excipient or additive. The pharmaceutical composition may contain other active agents. It may also be provided in a controlled release formulation or an immediate release formulation. The enantiomeric purity is preferably at least 90%, more preferably at least 95%, and most preferably at least 99%. In one embodiment, an oral dosage form contains about 0.1 to about 100 mg of (R,R′),(R,S′)-amphetaminil sulfate or another pharmaceutically-acceptable salt thereof; preferably, it contains about 1 to about 50 mg of (R,R′),(R,S′)-amphetaminil sulfate or another pharmaceutically-acceptable salt thereof.

In another aspect, the invention is directed to a method for the prophylaxis or treatment of a human condition or disease requiring or benefitting from a central stimulant by at least administering an effective amount of a pharmaceutical composition containing at least (R,R′),(R,S′)-amphetaminil sulfate or another pharmaceutically-acceptable salt thereof, substantially free of (S,R′),(S,S′)-amphetaminil. Administering may be, by way of non-limiting example, parenteral, transmucosal or transdermal. Non-limiting examples of transmucosal administration include orally, nasally, or rectally. Preferably, administration is oral. Non-limiting examples of parenteral administration include intra-arterial, intravenous, intramuscular, intradermal, subcutaneous, intraperitoneal, intraventricular, or intracranial. The amount administered may be about 0.1 to about 100 mg daily, preferably about 1 to about 50 mg daily. It may be administered in from one to about four unit doses per day, preferably in one or two unit doses per day.

The amount of (R,R′),(R,S′)-amphetaminil sulfate or another pharmaceutically-acceptable salt thereof in the pharmaceutical compositions or dosage forms of the invention is greater than about 90% of the weight of the total amphetaminil; preferably greater than about 95% of the weight of the total amphetaminil; and most preferably greater than about 99% of the weight of the total amphetaminil.

The amount of (R,R′),(R,S′)-amphetaminil sulfate or another pharmaceutically-acceptable salt thereof, substantially free of (S,R′),(S,S′)-amphetaminil, in a pharmaceutical composition or dosage form may be administered together with a pharmaceutically-acceptable carrier, diluent, excipient or additive. Other active agents may also be present.

By way of non-limiting examples, the conditions and diseases prophylaxed or treated with a pharmaceutical composition of dosage form of the invention includes narcolepsy, attention-deficit hyperactivity disorder (ADHD), depression, Parkinson's disease, cognitive dysfunction, Alzheimer's disease, renal dysfunction, hypotension, asthma, obesity, nicotine withdrawal, apathy, potentiating the activity of a conventional antidepressant, potentiating opiate activity for the treatment of pain, and decreased energy associated with chemotherapy or radiation treatment. It may be used for any condition or disease in which racemic amphetaminil, amphetamine, and more specifically, D-amphetamine, have been used. Moreover, less activation or promotion of stereotypic behavior is elicited by this composition than by racemic amphetaminil, and therefore may be less likely to exhibit or exacerbate movement disorders in patients in which this compound is administered. This is particularly beneficial to the treatment of Parkinson's disease and individuals with ADHD who exhibit tics.

It is a further object of the invention to provide for a method for the prophylaxis or treatment of a human condition or disease desirably benefiting from preferential activation of mesolimbic-mediated behaviour by at least administering to the human an effective amount of a pharmaceutical composition comprising (R,R′),(R,S′)-amphetaminil sulfate or another pharmaceutically-acceptable salt thereof, substantially free of (S,R′),(S,S′)-amphetaminil. Administering may be, by way of non-limiting example, parenteral, transmucosal or transdermal. Non-limiting examples of transmucosal administration include orally, nasally, or rectally. Preferably, administration is oral. Non-limiting examples of parenteral administration include intra-arterial intravenous, intramuscular, intradermal, subcutaneous, intraperitoneal, intraventricular, or intracranial. The amount administered is about 0.1 to about 100 mg daily, preferably about 1 to about 50 mg daily. It may be administered in from one to about four unit doses per day, preferably in one or two unit doses per day.

The amount of (R,R′),(R,S′)-amphetaminil sulfate or another pharmaceutically-acceptable salt thereof in the pharmaceutical compositions or dosage forms of this aspect of the invention is greater than about 90% of the weight of the total amphetaminil; preferably greater than about 95% of the weight of the total amphetaminil; and most preferably greater than about 99% of the weight of the total amphetaminil.

The amount of (R,R′),(R,S′)-amphetaminil sulfate or another pharmaceutically-acceptable salt thereof, substantially free of (S,R′),(S,S′)-amphetaminil, in a pharmaceutical composition or dosage form may be administered together with a pharmaceutically-acceptable carrier, diluent, excipient or additive. Other active agents may also be present.

These and other aspects of the present invention will be better appreciated by reference to the following drawings and Detailed Description.

### BRIEF DESCRIPTION OF THE DRAWINGS

FIGS. 1A, B and C depict the locomotor activity, cumulative locomotor activity and dose response curve, respectively, from an in-vivo study with amphetamine.

FIGS. 2A, B and C depict the locomotor activity, cumulative locomotor activity and dose response curve, respectively, from an in-vivo study with fusaric acid.

FIGS. 3A, B and C depict the locomotor activity, cumulative locomotor activity and dose response curve, respectively, from an in-vivo study with (R, R′),(R, S′)-amphetaminil.

FIGS. 4A, B and C depict the locomotor activity, cumulative locomotor activity and dose response curve, respectively, from an in-vivo study with (R, S′), (R, R′),(S, R′),(S, S′)-amphetaminil.

FIGS. 5A, B and C depict the stereotypy scores, total stereotypy score and dose response curve, respectively, from an in-vivo study with amphetamine

FIGS. 6A, B and C depict the stereotypy scores, total stereotypy score and dose response curve, respectively, from an in-vivo study with fusaric acid.

FIGS. 7A, B and C depict the stereotypy scores, total stereotypy score and dose response curve, respectively, from an in-vivo study with (R, R′),(R, S′)-amphetaminil.

FIGS. 8A, B and C depict the stereotypy scores, total stereotypy score and dose response curve, respectively, from an in-vivo study with (R, S′),(R, R′),(S, R′),(S, S′)-amphetaminil.

### DETAILED DESCRIPTION OF THE INVENTION

The present invention is directed to pharmaceutical compositions and methods of use of a form of amphetaminil substantially pure at the first dissymetric center with heretofore unrecognized and unexpected properties. As identified by the inventors herein, the (R,S′),(R,R′) form of amphetaminil ((R,S′),(R,R′)-amphetaminil, also referred to as alpha-[(1R-Methyl-2-phenylethyl)amino]benzeneacetonitrile]) possesses certain improved pharmacological and dose-responsive activities over that of racemic amphetaminil, and thus is useful in a pharmaceutical composition substantially free of (S,R′),(S,S′)-amphetaminil for the treatment of a number of conditions and diseases for which racemic amphetaminil, as well as other related compounds including amphetamine, and in particular D-amphetamine, have been used therapeutically. Moreover, reduced dosage and attendant reduced side effects or adverse effects or events are benefits of treating individuals with only an active form of amphetaminil for such indications, reducing manufacturing costs, size and packaging of dosage forms, and providing a smaller oral dosage form for better patient compliance.

The uses provided herein for a pharmaceutical composition comprising (R,S′),(R,R′)-amphetaminil embraces all of the uses heretofore identified for the racemate as well as that of other compounds in the same therapeutic category. As such, amphetaminil may be used for conditions for which treatment is indicated for a central nervous system stimulant. Such specific uses include but are not limited to narcolepsy, attention-deficit hyperactivity disorder (ADHD), depression, Parkinson's disease, cognitive dysfunction, Alzheimer's disease, renal dysfunction, hypotension, asthma, obesity, nicotine withdrawal, apathy, and decreased energy associated with chemotherapy or radiation treatment, optionally in combination with other agents. Such combinations include use with conventional antidepressants and opiates, for the treatment of depression and pain control, respectively. Theses uses are merely examples are not intended to be in any way limiting. For example, Martindale's reports that amphetaminil is prescribed as a central stimulant given by mouth in doses of 10 to 30 mg daily in the treatment of narcolepsy or narcoleptic syndrome. It is available from Krugmann GmbH (Limburg, Germany) under the name AN1, and in two formulations from Voigt GmbH (Limburg, Germany): Ton-O₂, containing heptaminol HCl, amphetaminil and adenosine indicated for treatment of hypotension, and Vit-O₂, containing amphetaminil and inositol nicotinate, indicated for use as a tonic.

The (R,R′),(R,S′) form of amphetaminil, based on the studies described herein, is particularly suited for the treatment of conditions and diseases in which preferential activity in the mesolimbic area of the brain is desirable, such as in the treatment of Parkinson's disease and in the ADHD patients who exhibit tics. As studies have shown efficacy in the treatment of ADHD in children using racemic amphetaminil (Pacit et al., 1996, Effect of Aponeuron in the treatment of children with hyperkinetic syndrome, Ceska Slov Psychiatr 92:41-57), the use of this form of amphetaminil may be desirable.

U.S. Pat. Nos. 6,166,032; 5,900,418; and 5,916,902 describe amphetaminil among other central nervous system stimulant for treatment of nicotine withdrawal, obesity, and for reducing the effects of antineoplastic disease treatment, respectively. All of the foregoing utilities are embraced herein.

The invention is directed, in one aspect, to a substantially pure pharmaceutical composition comprising (R,R′),(R,S′)-amphetaminil. Thus, the pharmaceutical composition of the invention comprises (R,R′),(R,S′)-amphetaminil substantially free of (S,R′),(S,S′)-amphetaminil. The terms “substantially free of (S,R′),(S,S′)-amphetaminil” as used herein means that the composition contain at least about 90% by weight of (R,R′),(R,S′)-amphetaminil and about 10% by weight or less of (S,R′),(S,S′)-amphetaminil. In a preferred embodiment the terms “substantially free of the (S,R′),(S,S′)-amphetaminil” mean that the composition contains at least 95% by weight of (R,R′),(R,S′)-amphetaminil and 5% or less of (S,R′),(S,S′)-amphetaminil. In a most preferred embodiment the terms “substantially free of (S,R′),(S,S′)-amphetaminil” means that the composition contains at least 99% by weight of (R,R′),(R,S′)-amphetaminil and 1% or less of (S,R′),(S,S′)-amphetaminil.

As mentioned above, amphetaminil possesses two dissymetric centers, one of which is part of the amphetamine-like portion of the molecule (and is the first to be set forth in the chemical structure), and the second part of the benzeneacetonitrile-like portion of the molecule (and is the second to be set forth, with a prime designation, in the chemical structure). The compound hereindescribed is the R form at the first or amphetamine-like dissymetric center, and is racemic at the second, or benzeneacetonitrile-like center. Thus, the amphetaminil compound of the invention may be described as (R,R′),(R,S′)-amphetaminil or synonymously as alpha-[(1R-methyl-2-phenylethyl)amino]-benzeneacetonitrile. Although the second dissymetric center may be racemic, it may be enriched in one form, or pure R′ or S′, without deviating from the teachings herein. Thus, the invention may also be extended to a pure amphetaminil enantiomer of (R,R′)-amphetaminil or (R, S′)-amphetaminil.

The Examples below provide a method for synthesis of (R,R′),(R,S′)-amphetaminil from chiral intermediates and stabilizing them as sulfate salts; however, any suitable method may be used to either synthesize the desired compound from starting materials, or to purify (R,R′),(R,S′)-amphetaminil [or the individual (R,R′) and (R,S′) enantiomers] from a racemic or other mixtures of amphetaminil of various ratios of stereoisomers. The invention is not so limiting as to the method of preparation, but embraces (R,R′),(R,S′)-amphetaminil effectively in the absence of (S,S′),(S,R′), to the extent possible based on cost, manufacturing, stability and other considerations. It will be apparent that several advantages are offered both to the patient and the manufacturer of the agent when (R,R′),(R,S′)-amphetaminil is used effectively in the absence of (S,S′),(S,R′), particularly for the conditions and diseases described herein, but it is not so limiting.

Moreover, while the sulfate salt of amphetaminil of the pharmaceutical composition of the invention is preferred, and without other reference amphetaminil referred to hereinthroughout is the sulfate salt (amphetaminil ½H₂SO₄), any pharmaceutically acceptable salt may be used. As will also be seen in the examples below, the sulfate salt was found to stabilize the R enantiomers during the synthesis procedure. The invention is also directed to a facile means for stabilizing the enantiomers by converting the products of the synthetic reaction at the stage of formation of amphetaminil into sulfate salts.

The pharmaceutical composition of the composition comprising (R,R′),(R,S′)-amphetaminil substantially free of (S,R′),(S,S′)-amphetaminil may be formulated in any pharmaceutically-acceptable carrier or excipient suitable for the route of administration and dosing frequency desired. Preferably, an oral formulation is provided, comprising the (R,R′),(R,S′)-amphetaminil in a tablet or capsule. However, formulations for other routes of administration are fully embraced herein, and the appropriate means for formulation will be determinable by the skilled artisan. Formulations for injection, for example, are also embraced herein.

According to the invention, the pharmaceutical composition of the invention may be introduced parenterally or transmucosally, e.g., orally, nasally, or rectally, or transdermally. Preferably, administration is oral. Examples of parenteral administration include intra-arterial, intravenous, intramuscular, intradermal, subcutaneous, intraperitoneal, intraventricular, and intracranial administration.

In yet another aspect of the present invention, provided are pharmaceutical compositions of (R,R′),(R,S′)-amphetaminil. Such pharmaceutical compositions may be for administration for injection, or for oral, pulmonary, nasal or other forms of administration. In general, comprehended by the invention are pharmaceutical compositions comprising effective amounts of a low molecular weight component or components, or derivative products, of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present amphetaminil composition. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form.

In the case of oral delivery, contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the component or components (or chemically modified forms thereof) and inert ingredients which allow for protection against the stomach environment, and release of the biologically active material in the intestine.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

One may dilute or increase the volume of the therapeutic with an inert material. These diluents could include carbohydrates, especially mannitol, alpha-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 or 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the amphetaminil either alone or as a mixture in different ratios.

Controlled release oral formulation may be desirable. The drug could be incorporated into an inert matrix which permits release by either diffusion or leaching mechanisms, e.g., gums. Slowly degenerating matrices may also be incorporated into the formulation. Some enteric coatings also have a delayed release effect.

Another form of a controlled release of this therapeutic is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan-coater or in a fluidized bed or by compression coating.

The foregoing provides mere examples of dosage forms of the present invention. Moreover, (R,R′),(R,S′)-amphetaminil may be formulated in combination with one or more other pharmacologically active agents or compounds that are necessary or desirable for achieving a desired pharmacological effect.

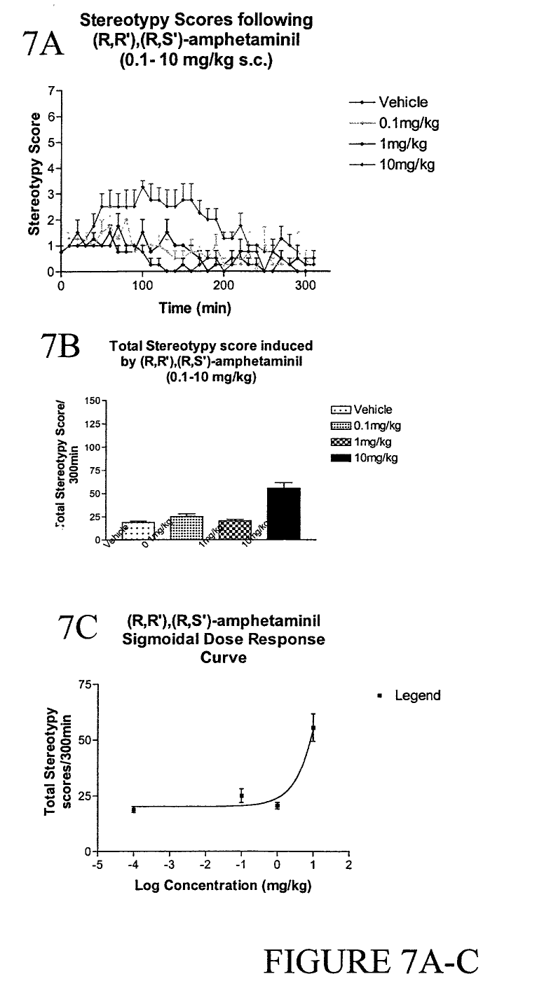

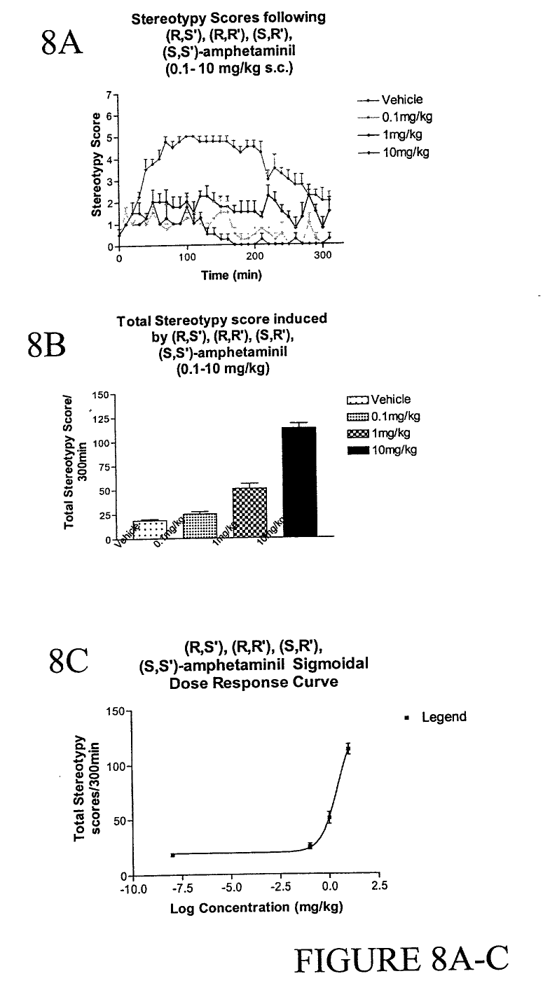

The dosage of (R,R′),(R,S′)-amphetaminil will be readily determined for the particular indication. For example, racemic amphetaminil is administered at 10 to 30 mg per day for the treatment of narcolepsy. Based on the studies herein, and the identification of the (R,R′),(R,S′) compound as possessing the pharmacological activity of the racemate, a dosage of 1 to about 50 mg per day is provided. However, as each condition to be treated required a readily-determinable dose amount and frequency to achieve the desired effect, this dosage is not so limiting, and doses from 0.1 mg up to about 100 mg are included herein. As is noted in FIGS. 7 and 8 herein, the (R,R′),(R,S′) form exhibits less stereotypy than the racemic form of amphetaminil.

In a further, and theoretical analysis of the results obtained herein of which Applicants have no duty to disclose and are not bound thereby, the effect on stereotypy activity of the R form of amphetaminil indicates that it preferentially activates mesolimbic-mediated behavior, and as such is useful for treatment of conditions and diseases mediated through this part of the brain. Non-limiting examples of such conditions and diseases are set forth herein, yet the invention embraces all such conditions and diseases in which a preferentially mesolimbically-active compound is therapeutically useful.

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLE 1

### Synthesis of Amphetaminil and Analytical Summary

In preliminary studies, (+/−)-amphetaminil free base was synthesized. It was purified by recrystallization to form a white crystalline solid. It was fully characterized; high field proton NMR in deuterochloroform showed a 5:1 ratio of diastereomers, but was clean with respect to other impurities.

The (+) and (−) amphetaminils were then synthesized. These materials did not recrystallize successfully from a variety of solvents tested. When any solid was isolated, it rapidly liquefied at room temperature, consequently adequate purity could not be obtained by this purification method. It was reasoned that the different behavior from the racemate was due to the fact that enantiomerically pure materials can have different crystallization properties from racemates, and will usually form lower melting solids. Next, purification was attempted by formation of the HCl salt, followed by recrystallization and free-up back to amphetaminil. This was unsuccessful, and showed almost complete breakdown for all isomers.

Chromatographic purification of the (+) and (−) amphetaminil free bases was then attempted, including silica gel, basic alumina, neutral alumina as well as deactivated alumina and silica. Preparatory TLC was also attempted. All attempts led to very poor recoveries and lower purities, indicating degradation, and no separation of diastereomers was noted. Due to poor reverse phase HPLC results, reverse phase preparative chromatography was not attempted. Normal Phase preparative purification using a chiral HPLC Column also led to degradation.

A larger batch of (+)-amphetaminil was synthesized. Further experiments with recrystallization of the free base were performed, but no improvements over the above were found. In subsequent studies, various salts were used. The sulfate salts gave the most promising results, with little breakdown.

The (+) and (−) amphetaminils were resynthesized to insure highest purity compounds and converted to the sulfate salts immediately. Some further experimentation with the sulfate salt showed it to be somewhat heat-sensitive, so rather than recrystallizing, the salt was purified by trituration (washing) with solvent (ethanol/ether mix) to remove impurities. It was found that the impurities formed during the synthesis could be removed, but that a new impurity began to form as washing continued. Stopping at the optimal point resulted in 3 products that were 98-99% pure by gas chromatography. The (+/−) material, which was the most pure, was subjected to further analytical testing.

In order to insure a consistent ratio of the diastereomers, the racemic material was resynthesized from racemic amphetamine sulfate previously obtained. It was immediately made in to the sulfate salt without any recrystallization and purified by trituration as with the (+) and (−) isomers.

A chiral analytical column proved to be more successful in separation of the isomers of amphetaminil. Sample preparation involved freeing up the sulfate with an ammonium hydroxide solution followed by extraction with hexane. The sample would then be injected. Even with rapid injection, breakdown was evident by the formation of a large benzaldehyde peak that would increase with time as the sample solution sat around. However, consistent separations of all 4 isomers was obtained, with the following results:

| | | | (R,S′)- | (S,R′)- |
|---|---|---|---|---|
| | (R,R′)- | (S,S′)- | Ampheta- | Ampheta- |
| | Amphetaminil | Amphetaminil | minil | minil |
| CSQ-1680A | 50% | 0% | 50% | 0% |
| CSQ-1680E | 25% | 25% | 25% | 25% |

This was in contrast to the result obtained by NMR for the racemic recrystallized amphetaminil, which showed a 4-5:1 ratio in the original analysis of the free base.

These results were confirmed by NMR analysis. Based on the information we learned about the stability of amphetaminil in various solvents, we obtained analyses of the sulfate in acetone-D6 as well as freed up sample in deuterochloroform. Various degrees of degradation were seen in these NMRs, but the ratio in all three products closely matched the results obtained from chiral LC analysis. An accurate measurement of the ratio of isomers is difficult due to the instability of the material in solution and the analytical variability in NMR analysis, but an averaging of our results resulted in the ratios indicated in the table above.

### EXAMPLE 2

### Synthesis Procedure for (R, R′),(R,S′)-Amphetaminil Sulfate; CSQ-1680A

Step 1—(1S, 2R)-(+)-norephedrine hydrochloride

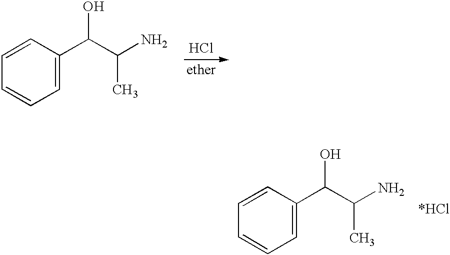

The HCl salt of (1S, 2R)-(+)-norephedrine was made by bubbling HCl gas into a flask containing (1S, 2R)-(+)-norephedrine (200 grams) in ethyl ether (500 mL). The salt was filtered, washed with ethyl ether, and dried to yield 150 g of (1S, 2R)-(+)-norephedrine hydrochloride.

Step 2—(1R, 2R)-norchloroephedrine hydrochloride

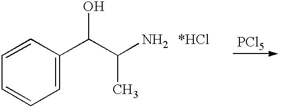

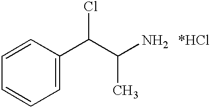

| | Formula | Mass | | | Vendor and |
|---|---|---|---|---|---|
| Compound | Weight | Used | Moles | Equivalents | Lot# |
| Phosphorus pentachloride | 208.24 | 43.7 g | 0.21 | 1.3 | Aldrich |
| | | | | | 13713PS |
| (1S,2R)-(+)-Norephedrine | 187.67 | 30.0 g | 0.16 | 1.0 | Aldrich |
| hydrochoride | | | | | 01233JF |

(1S, 2R)-(+)-norephedrine hydrochloride was added portionwise to a 500 mL round bottom flask containing phosphorus pentachloride in chloroform (300 mL) and was stirred at room temperature under nitrogen overnight. The norchloroephedrine salt that precipitated out of the reaction was filtered, washed with chloroform, and dried to yield 22.4 grams (68% yield).

Step 3—(−)-amphetamine

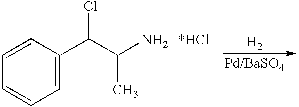

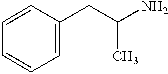

| | Formula | Mass | | | | |
|---|---|---|---|---|---|---|
| Compound | Weight | Used | | Moles | Equivalents | Vendor and Lot# |
| Pd on BaSO4 | | 11.8 | g | | | Aldrich 08825JR |
| Norchloroephedrine | 206.17 | 22.4 | | 0.11 | 1 | Product of step 2 |
| HCl | | | | | | |
| Sodium Acetate | 136.08 | 58.8 | g | 0.43 | 4 | Mallincrodt |
| trihydrate | | | | | | 7364KETT |

A suspension of the sodium acetate, 5% palladium on Barium sulfate and racemic norchloroephedrine hydrochloride in 186 mL glacial acetic acid and 9.7 mL deionized water was hydrogenated at 50 psi for 72 h. The reaction was then filtered through celite and the celite washed with water. The majority of the acetic acid was removed in vacuo and the pH adjusted to 10 with 10% sodium hydroxide solution. After extracting three times with ethyl ether, the combined organics were dried over anhydrous sodium sulfate. The ether was then removed by fractional distillation at ambient pressure, followed by fractional distillation at 1 mm Hg to yield 11.0 g (74%) of (−)-amphetamine at 99% purity by Gas Chromatography.

Step 4—(R, R′)-Amphetaminil Sulfate and (R,S′)-Amphetaminil Sulfate

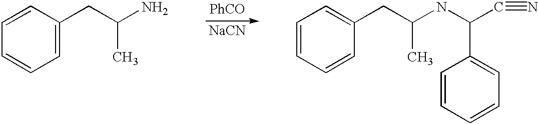

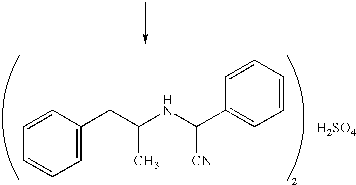

| | Formula | Mass | | | | Vendor and |
|---|---|---|---|---|---|---|
| Compound | Weight | Used | | Moles | Equivalents | Lot# |
| Amphetamine | 135.21 | 10.43 | | 0.077 | 1.0 | Product of step |
| | | | | | | 3 |
| Benzaldehyde | 106.12 | 8.13 | g | 0.077 | 1.0 | Aldrich |
| | | | | | | 00912LQ |
| Sodium cyanide | 49.01 | 3.38 | g | 0.069 | 0.9 | Aldrich |
| | | | | | | 00426KB |

The amphetamine was suspended in 11 mL water. The solution was adjusted to pH7 with 10% sulfuric acid. A solution of the sodium cyanide in 11 mL water was then added. The solution went clear. The benzaldehyde dissolved in 27 mL methanol was then added over a 10-minute period causing a slight evolution of heat. After stirring for 1h there was no amphetamine left by GC. The reaction mixture was extracted three times with ethyl ether and the combined organics dried over anhydrous sodium sulfate and then filtered. The sulfate salt was immediately made by adding a solution of sulfuric acid and ethyl ether dropwise. The salt was filtered, washed and dried to yield 2.8 grams of a 1:1 mixture of (R, R′)-Amphetaminil Sulfate and (R,S′)-Amphetaminil Sulfate.

### EXAMPLE 3

### Synthesis Procedure for (R,R′), (S,S′), (R,S′), (S,R)-Amphetaminil Sulfate, CSQ-1680E

Step 1—(1S, 2S), (1R,2R)-norchloroephedrine hydrochloride

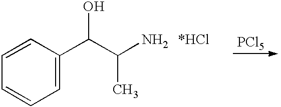

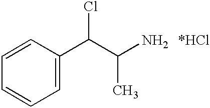

| | Formula | Mass | | | | Vendor and |
|---|---|---|---|---|---|---|
| Compound | Weight | Used | | Moles | Equivalents | Lot# |
| Phosphorus pentachloride | 208.24 | 73.1 | g | 0.35 | 1.3 | Aldrich |
| | | | | | | 06011BS |
| (1S,2R),(1R,2S)Norephedrine | 187.67 | 50.0 | | 0.27 | 1.0 | Aldrich |
| hydrochloride | | | | | | 13713RS |

A 1 L round bottomed flask was charged with the phosphorus pentachloride and 500 mL anhydrous chloroform. To this stirring mixture under nitrogen was added portion-wise the Norephedrine hydrochloride. The reaction was stirred for 15 h after which the precipitate that formed was filtered and washed with chloroform, then dried under high vacuum to yield 47.4 g (86%) of a white solid. This material was taken on without further purification.

Step 2—(+/−)-Amphetamine

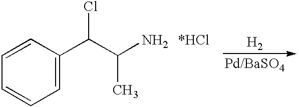

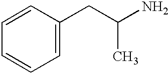

| | Formula | Mass | | | | |
|---|---|---|---|---|---|---|
| Compound | Weight | Used | | Moles | Equivalents | Vendor and Lot# |
| Pd on BaSO4 | | 10.5 | g | | | Aldrich 08825JR |
| Norchlorephedrine | 206.17 | 20.0 | | 0.1 | 1 | Product of prior |
| | | | | | | step |
| Sodium Acetate | 136.08 | 87 | g | 0.64 | | Mallincrodt |
| trihydrate | | | | | | 7364KETT |

A suspension of the sodium acetate, 5% palladium on Barium sulfate and racemic norchlorepedrine hydrochloride in 166 mL glacial acetic acid was hydrogenated at 50 psi for 24 h. The reaction was filtered through celite and the celite washed with water. The acetic acid was removed in vacuo and the pH adjusted to 10 with 10% sodium hydroxide solution. The aqueous solution was extracted three times with ethyl ether and the combined organics dried over anhydrous sodium sulfate. The ether was then removed by fractional distillation at ambient pressure, followed by amphetamine distillation at 1 mm Hg to yield 10.0 g (76%) at 99% purity by Gas Chromatography.

Step 3—(S, S′)-Amphetaminil Sulfate, (S, R′)-Amphetaminil Sulfate, (R, R′)-Amphetaminil Sulfate, (R, S′)-Amphetaminil Sulfate

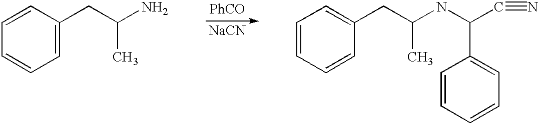

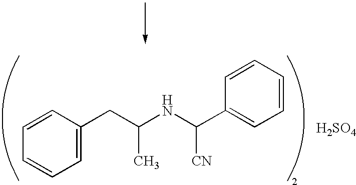

| | Formula | Mass | | | | Vendor and |
|---|---|---|---|---|---|---|
| Compound | Weight | Used | | Moles | Equivalents | Lot# |
| Amphetamine | 135.21 | 10.0 | | 0.074 | 1.0 | Product of |
| | | | | | | prior step |
| Benzaldehyde | 106.12 | 7.73 | g | 0.073 | 1.0 | Aldrich |
| | | | | | | 00912LQ |
| Sodium cyanide | 49.0 | 2.7 | g | 0.055 | 0.75 | Aldrich |
| | | | | | | 00426KB |

The amphetamine was suspended in 11 mL water. The solution was adjusted to pH 7 with 10% sulfuric acid. A solution of the sodium cyanide in 11 mL water was then added. The solution went clear. The benzaldehyde dissolved in 27 mL methanol was then added over a 10-minute period causing a slight evolution of heat. After stirring for 1 h there was no amphetamine left by GC. The reaction mixture was extracted three times with ethyl ether and the combined organics dried over anhydrous sodium sulfate. Removal of solvent yielded an oil that was precipitated as a solid by the addition of ethanol followed by water. The solid was then recrystallized twice from ethanol/water to yield white crystals.

### EXAMPLE 4

### Effect of (R, R′),(R,S′)-Amphetaminil Sulfate (CSQ-1680A) and (R,R′), (S,S′), (R,S′), (S,R)-Amphetaminil Sulfate (CSQ-1680E) on Locomotor Activity and the Induction of Stereotyped Behaviour in Normal Rats: Comparison with Amphetamine and Fusaric Acid

Male Wistar rats (n=4 per group) were randomly allocated to each drug treatment group. Drugs were administered such that, with the exception of fusaric acid, each rat in each group received all doses of the drug or vehicle in a semi random manner according to the modified Latin Square in Table 1. Compounds were administered at the doses: 0.1, 1 and 10 mg/kg s.c. in 100% DMSO (vehicle). Amphetamine was administered at 0.1, 1 and 5 mg/kg s.c. in saline (vehicle). Fusaric acid was administered at 20, 40 and 80 mg/kg s.c. in saline (vehicle). Rats received each dose of fusaric acid (20, 40 and 80 mg/kg), doses given in order, starting with vehicle. A one-week interval was provided between doses.

Table 1 depicts a modified Latin Square for amphetamine and the amphetaminil compositions used in the in-vivo studies. Rats received the following treatments: 1-4, Amphetamine; 9-12, (R, R′),(R, S′)-amphetaminil; and 13-16, (R, S′),(R, R′),(S, R′),(S, S′)-amphetaminil.

TABLE I

| | Week | | | |
|---|---|---|---|---|
| Rat | 1 | 2 | 3 | 4 |
| 1/9/13 | A | C | D | B |
| 2/10/14 | D | B | A | C |
| 3/11/15 | C | A | D | B |
| 4/12/16 | B | C | D | A |

Key to Table 1

| | | Amphetaminil |
|---|---|---|
| Treatments | Amphetamine | composition |
| A | 5 mg/kg | 0.1 mg/kg |
| B | 1 mg/kg | 1 mg/kg |
| C | 0.1 mg/kg | 10 mg/kg |
| D | control | Control |

At the start of the experiment, rats were placed in standard perspex behaviour boxes with a visible grid on the floor to allow determination of locomotor activity. The animals were allowed to acclimatise to their environment for 1 hour prior to drug/saline administration. Following drug administration animals were returned to the behaviour cages and locomotor activity was assessed for a further 5 hours or until baseline (zero activity for 30 minutes) was achieved, whichever was sooner. Locomotor activity was assessed by videoing the rats throughout the experiment, and counting the number of times a rat crossed a grid line over a period of 5 minutes every 10 minutes. Throughout the experiment animals were scored for stereotyped behaviours for 15 seconds every 10 minutes by a trained observer using the scoring methods described in Table 2.

TABLE 2
Stereotypy Scoring

| Number | Behaviour |
|---|---|
| 0 | Asleep |
| 1 | Inactive |
| 2 | Normal activity/grooming |
| 3 | Sniffing/rearing |
| 4 | Continuous sniffing and rearing, moving along fixed path |
| 5 | Continuous sniffing and rearing, staying in one location |
| 6 | Continuous Stereotypy, licking and gnawing |
| 7 | Continuous licking and gnawing |

Data Analysis

Locomotor activity Data The mean and standard error for locomotor activity during 10 minutes for each rat at each time point was determined. This data was analysed by two-way ANOVA for matched samples (GraphPad Prizm). Cumulative activity during the 300 min was also calculated, and the effect of dose of drug was analyzed by 1 way repeated measures ANOVA followed by Dunnett's test (GraphPad Prizm). Data was also analysed for significant fit to a sigmoidal-shaped log dose-response curve using non-linear regression analysis (GraphPad Prizm).

Stereotypy Score Data

The mean and standard error for stereotypy activity for 15 seconds every 10 minutes for each rat at each time point was determined. This data was analysed by two-way ANOVA for matched samples (GraphPad Prizm). Cumulative activity during the 300 min was also calculated, and the effect of dose of drug was analyzed by Kruskall Wallis one-way ANOVA (GraphPad Prizm). Data was also analysed for significant fit to a sigmoidal-shaped log dose-response curve using non-linear regression analysis (GraphPad Prizm)

Results

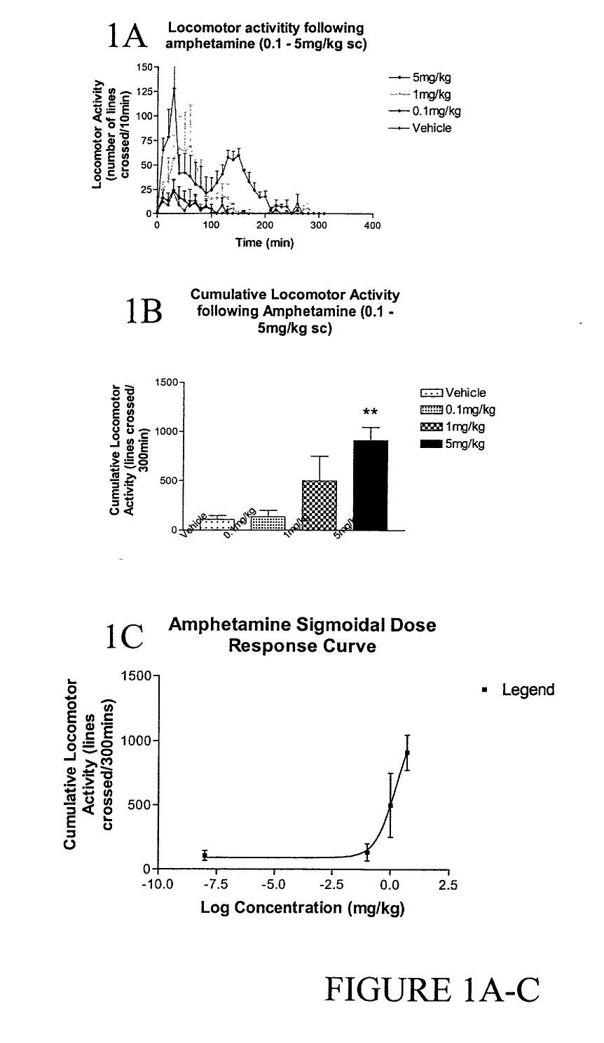

Locomotor Activity Locomotor activity for amphetamine (0.1-5 mg/kg sc); fusaric acid (20-80 mg/kg sc); (R, R′),(R, S′)-amphetaminil (0.1-10 mg/kg sc); and (R, S′), (R, R′),(S, R′),(S, S′)-amphetaminil) (0.1-10 mg/kg sc) are shown in FIGS. 1-4, respectively. Panel A of each figure shows the locomotor activity recorded over time, for each dose and vehicle; panel B the cumulative locomotor activity for each dose; and panel C a dose response.

Amphetamine (FIG. 1) produced a dose-related increase in locomotor activity although data did not fit a sigmoidal-shaped log dose-response curve (FIGS. 1A-C). Amphetamine (5 mg/kg) significantly increased cumulative locomotor activity during the 300 min duration of the experiment (FIG. 1A; significant effect of time (p<0.001), treatment (p<0.001), and interaction (p<0.001), 2-way ANOVA). Onset of activity at 5 mg/kg was rapid, with maximum effect seen 30 min after administration. Data represents mean±SEM of 4 rats, each rat receiving each dose in a random manner over 4 weeks. In the cumulative figure, **p<0.01 compared to vehicle (Dunnett's t-test). The data does not fit a sigmoidal-shaped log dose-response relationship (R=0.788).

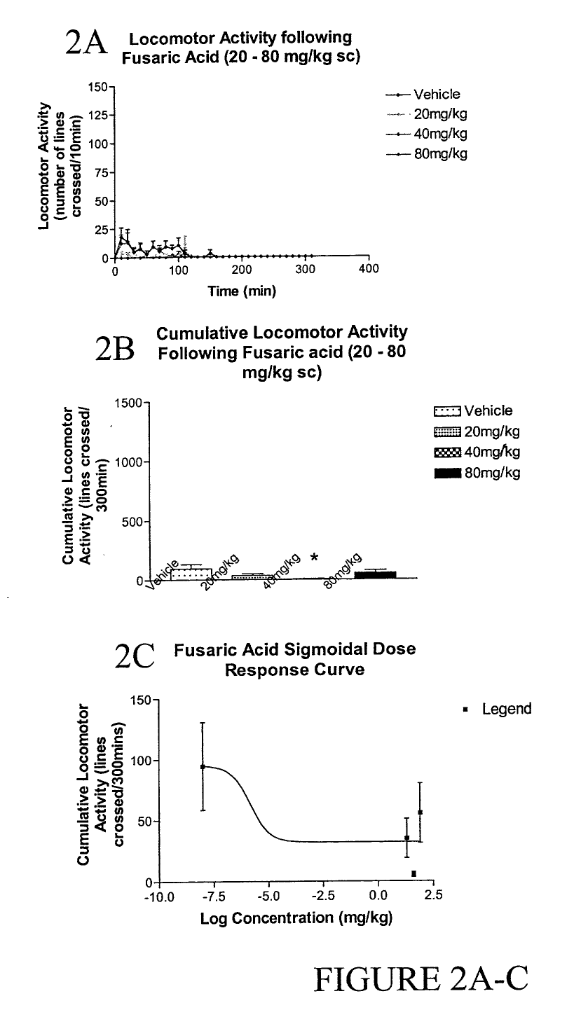

Fusaric acid (FIG. 2) did not increase locomotor activity, however, a small, but significant inhibition of normal activity was observed after administration of 40 mg/kg, but not 80 mg/kg (FIGS. 2A-B; significant effect of time (p<0.001), treatment (p<0.001), and interaction (p<0.001), two-way ANOVA). In the cumulative locomotor counts during the 300 minutes post-injection in normal rats, * p<0.05 compared to vehicle (Dunnett's t-test). These data did not fit a sigmoidal-shaped log dose-response relationship (FIG. 2C).

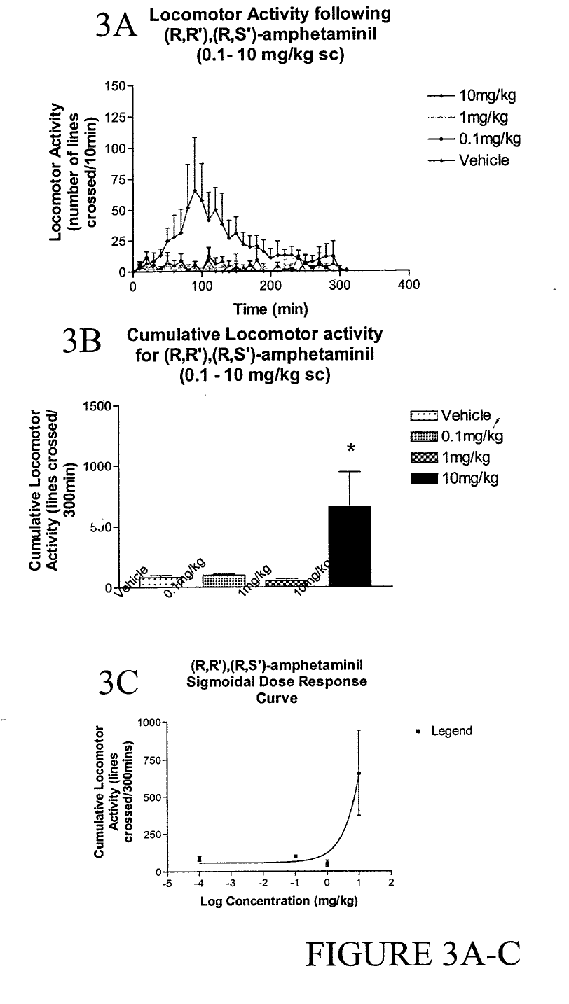

(R, R′),(R, S′)-Amphetaminil (FIG. 3) increased locomotor activity only at the highest dose of 10 mg/kg (FIGS. 3A-B; significant effect of treatment (p<0.001), two-way ANOVA). In the cumulative graph, * p<0.05 compared to vehicle (Dunnett's t-test). These data did not fit a sigmoidal-shaped log dose-response relationship (FIG. 3C).

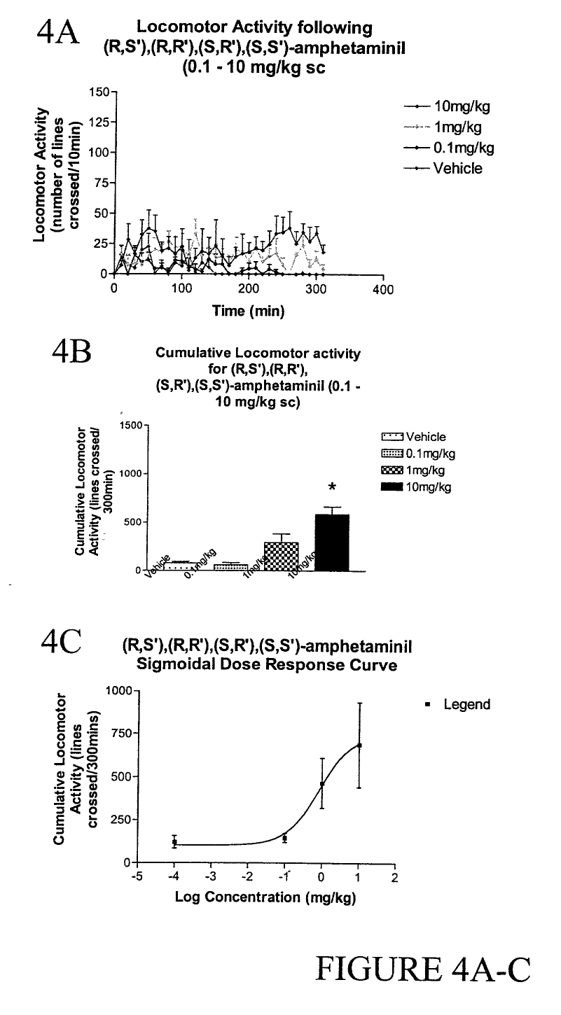

Racemic amphetaminil [(R, S′), (R, R′),(S, R′),(S, S′)-amphetaminil] (FIG. 4) increased locomotor activity only at the highest dose of 10 mg/kg (FIGS. 4A-C). In the cumulative graph, * p<0.05 compared to vehicle (Dunnett's t-test). These data did not fit a sigmoidal-shaped log dose-response relationship (R=0.6836) (FIG. 4C).

Stereotypy Analyses.

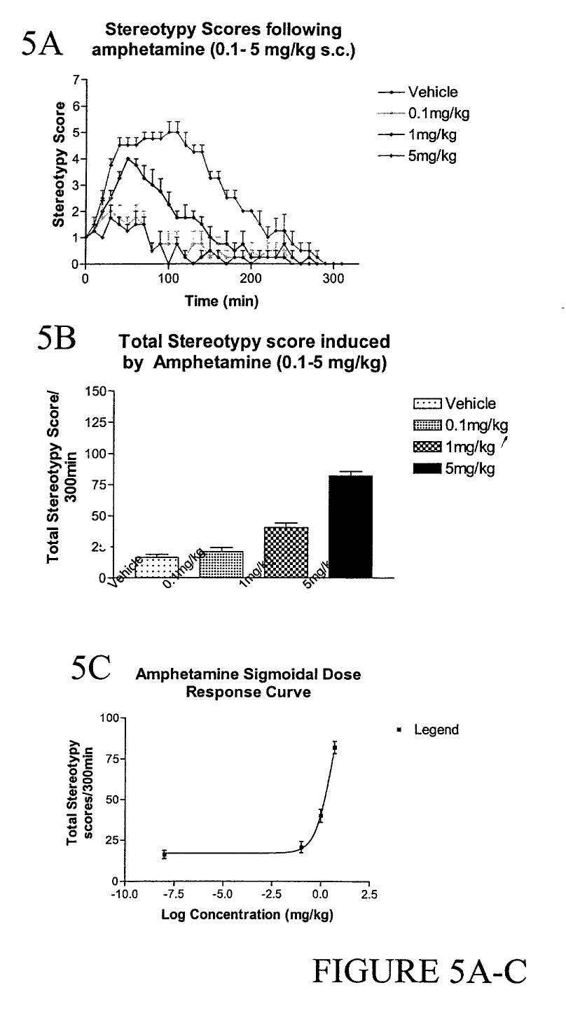

Stereotypy scores for amphetamine (0.1-5 mg/kg sc), fusaric acid (20-80 mg/kg sc), (R, R′),(R, S′)-amphetaminil (0.1-10 mg/kg sc), and (R, S′), (R, R′),(S, R′),(S, S′)-amphetaminil) (0.1-10 mg/kg sc) are shown in FIGS. 5-8. In each figure, panel A shows the stereotypy scores over time for each dose, panel B the total stereotypy score for each dose, and panel C the dose-response curve.

Amphetamine (FIG. 5) produced a log dose-related increase stereotypy (FIGS. 5A-B; Significant effect of time (p<0.001), treatment (p<0.001), and interaction (p<0.001), two-way ANOVA(matching)). Amphetamine (5 mg/kg) tended to increase overall stereotypy during the 300 min duration of the experiment; data fitted a sigmoidal-shaped log dose-response curve p<0.001, R=0.9742.

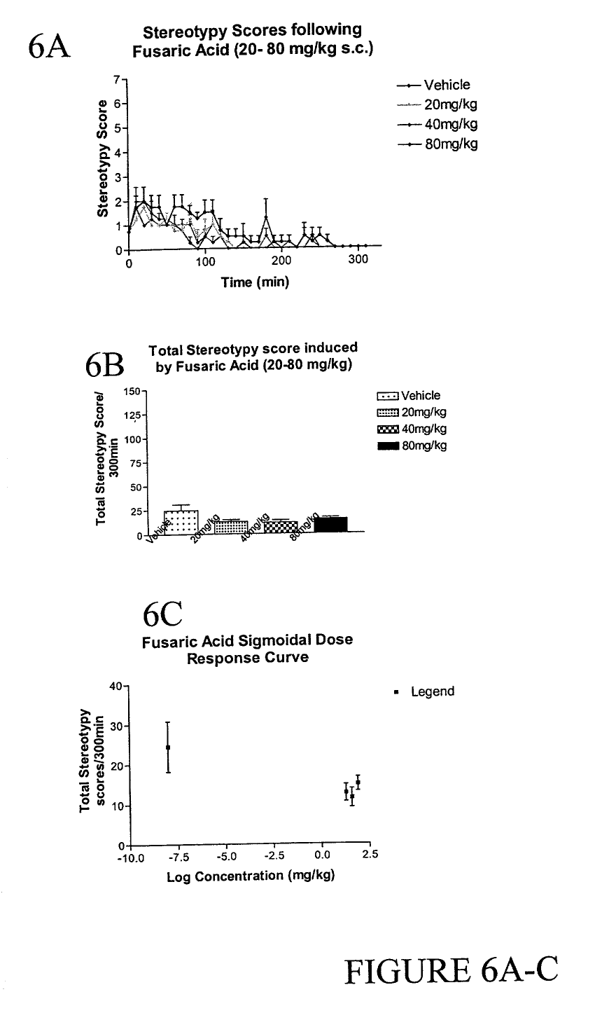

Fusaric acid (FIG. 6) did not increase stereotypy (FIGS. 6A-B). However, the animals tended to sleep more following fusaric acid, although this was not statistically significant (p>0.04, Kruskall-Wallis).

(R, R′),(R, S′)-Amphetaminil (FIG. 7) tended to increase stereotypy at 10 mg/kg (FIGS. 7A-C) although differences did not reach statistical significance (p>0.05, Kruskall-Wallis). The data does not fit a sigmoidal-shaped dose-response relationship.

Racemic amphetaminil [(R, S′), (R, R′),(S, R′),(S, S′)-amphetaminil] (FIG. 8) increased stereotypy in a log dose-related manner (FIGS. 8A-C). Onset of stereotypy was rapid, and, following 10 mg/kg, stereotypy was sustained for the duration of the experiment, peaking between 100 and 200 min (Significant effect of time (p<0.001), treatment (p<0.001), and interaction (p<0.001), two-way ANOVA(matching)). However, differences in overall stereotypy between groups did not reach statistical significance (FIG. 8C). The data fits a sigmoidal-shaped log dose-response relationship, p<0.05, R=0.9834.

It was found that the R form of the compound exhibited a higher ratio at both the time of peak effect and total effect versus that of the racemic amphetaminil, as set forth below.

| | Loco- | Stereo- | | Loco- | Stereo- | |
|---|---|---|---|---|---|---|
| | motor | typy | Ratio | motor | typy | Ratio |
| Compound | (Peak) | (Peak) | (Peak) | (Total) | (Total) | (Total) |
| (R,R′), (R,S′)- | 62 | 3.3 | 18.8 | 625 | 57 | 11 |
| amphetaminil | | | | | | |
| Racemic | 37 | 5.0 | 7.4 | 560 | 115 | 4.9 |
| amphetaminil | | | | | | |
| Amphetamine | 125 | 5 | 25 | 850 | 81 | 10.5 |

The R form thus may be less likely to elicit or exacerbate movement disorders in patients, regardless of the indication for which this compound may be administered. As noted above, tics are prevalent in about 12% of ADHD patients, and thus the R form of amphetaminil may be better suited for such treatment than the racemate.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. A pharmaceutical composition comprising an effective amount of (R,R′),(R,S′)-amphetaminil sulfate or another pharmaceutically-acceptable salt thereof, substantially free of (S,R′),(S,S′)-amphetaminil, and at least one pharmaceutically-acceptable carrier, diluent, excipient or additive.

2. A controlled release formulation comprising the pharmaceutical composition of claim 1.

3. An immediate release formulation comprising the pharmaceutical composition of claim 1.

4. An oral dosage form comprising the pharmaceutical composition of claim 1 consisting of about 0.1 to about 100 mg of (R,R′),(R,S′)-amphetaminil sulfate or another pharmaceutically-acceptable salt thereof.

5. The dosage form of claim 4 consisting of about 1 to about 50 mg of (R,R′),(R,S′)-amphetaminil sulfate or another pharmaceutically-acceptable salt thereof.

6. The pharmaceutical composition of claim 1 wherein said (R,R′),(R,S′)-amphitaminil sulfate or another pharmaceutically-acceptable salt thereof is greater than about 90% of the weight of total amphetaminil.

7. The pharmaceutical composition of claim 6 wherein said (R,R′),(R,S′)-amphetaminil sulfate or another pharmaceutically-acceptable salt thereof is greater than about 95% of the weight of total amphetaminil.

8. The pharmaceutical composition of claim 7 wherein said (R,R′),(R,S′)-ampetaminil sulfate or another pharmaceutically-acceptable salt thereof is greater than about 99% of the weight of total amphetaminil.

9. A method for prophylaxis or treatment of a human condition or disease requiring or benefitting from a central nervous stimulant comprising administering to said human an effective amount of a pharmaceutical composition comprising (R,R′),(R,S′)-amphetaminil sulfate or another pharmaceutically-acceptable salt thereof, substantially free of (S,R′),(S,S′)-amphetaminil.

10. The method of claim 9 wherein said administering is parenteral, transmucosal or transdermal.

11. The method of claim 10 wherein said transmucosal is orally, nasally, or rectally.

12. The method of claim 10 wherein said parenteral is intra-arterial, intravenous, intramuscular, intradermal, subcutaneous, intraperitoneal, intraventricular, or intracranial.

13. The method of claim 9 wherein the amount administered is about 0.1 to about 100 mg daily.

14. The method of claim 13 wherein said amount administered is about 1 to about 50 mg daily.

15. The method of claim 14 wherein the amount is administered from one to about four unit doses per day.

16. The method of claim 15 wherein the amount administered is one or two unit doses per day.

17. The method of claim 5 wherein the amount of (R,R′),(R,S′)-amphetaminil sulfate or another pharmaceutically-acceptable salt thereof is greater than about 90% of the weight of the total amphetaminil.

18. The method of claim 17 wherein the amount of (R,R′),(R,S′)-amphetaminil sulfate or another pharmaceutically-acceptable salt thereof is greater than about 95% of the weight of the total amphetaminil.

19. The method of claim 18 wherein the amount of (R,R′),(R,S′)-amphetaminil sulfate or another pharmaceutically-acceptable salt thereof is greater than about 99% of the weight of the total amphetaminil.

20. The method of claim 9 wherein said amount of (R,R′),(R,S′)-amphetaminil sulfate or another pharmaceutically-acceptable salt thereof, substantially free of (S,R′),(S,S′)-amphetaminil is administered together with a pharmaceutically-acceptable carrier, diluent, excipient or additive.

21. The method of claim 9 wherein said condition or disease is narcolepsy, attention deficit hyperactivity disorder (ADHD), depression, Parkinson's disease, cognitive dysfunction, or Alzheimer's disease, renal dysfunction, asthma, obesity, nicotine withdrawal, hypotension, apathy, potentiating activity of a conventional antidepressant, potentiating an opiate for pain control, or reduced energy associated with chemotherapy or radiation therapy.

22. The method of claim 9 wherein said condition or disease is amenable to treatment by preferential activation of mesolimbic-mediated behavior.
